(19) United States
(12) Patent Application Publication
(10) Pub. No.: US 2003/0083273 A1
(43) Pub. Date: May 1, 2003

(54) ANTISENSE OLIGOMERS

(76) Inventor: TOD M. WOOLF, SUDBURY, MA (US)

Correspondence Address: LAHIVE & COCKFIELD, 28 STATE STREET, BOSTON, MA, 02109, US

(21) Appl. No.: 09/295,189

(22) Filed: Apr. 20, 1999

(51) Int. Cl. A61K 48/00; C07H 21/04

(52) U.S. Cl. 514/44; 536/23.2

(57) ABSTRACT

Antisense oligomers which possess improved properties over those taught in the prior art are disclosed. The instant methods enable the enhanced uptake of oligomers, increased affinity of the oligomers for their target molecules, increased resistance of oligomers to nucleases, decreased toxicity. The invention provides optimized antisense oligomer compositions and method for making and using the both in in vitro systems and therapeutically. The invention also provides methods of making and using the improved antisense oligomer compositions.

### BACKGROUND OF THE INVENTION

Antisense oligomers are promising therapeutic agents and useful research tools in elucidating gene function.

One established mechanism of antisense inhibition is the RNase H mediated cleavage of a target oligomer through cleavage of the RNA strand in DNA/RNA hybrids. It has been demonstrated that phosphorothioate DNA functions by activating endogenous RNase H and thereby cleaving the targeted RNA (Agrawal, S., Mayrand, S. H., Zamecnik, P. C. & Pederson, T. Proc Natl Acad Sci USA 87, 1401-5 (1990): Woolf, T. M., Jennings, C. G., Rebagliati, M. & Melton, D. A. Nucleic Acids Res 18, 1763-9 (1990)). With the notable exception of phosphorothioate DNA, the vast majority of nuclease resistant modified DNA backbones are not recognized by RNase H. While phosphorothioate DNA has the advantage of activating RNase H, phosphorothioate DNA has the disadvantage of non-specific effects and reduced affinity for RNA (Stein, C.A., Matsukura, M., Subasinghe, C., Broder, S. & Cohen, J. S. Aids Res Hum Retroviruses 5, 639-46 (1989): Woolf, T. M., Jennings, C. G., Rebagliati, M. & Melton, D. A. Nucleic Acids Res 18, 1763-9 (1990).

Gapmer or chimeric antisense oligomers that have a short stretch of phosphorothioate DNA (5-12 nucleotides) have been used to obtain RNase-H mediated cleavage of target RNAs, while reducing the number of phosphorothioate linkages (Dagle, J. M., Walder, J. A. & Weeks, D. L. Nucleic Acids Res 18, 4751-7 (1990); Agrawal, S., Mayrand, S. H., Zamecnik, P. C. & Pederson, T. Proc Natl Acad Sci U S A 87, 1401-5 (1990).) Usually, in a gapmer oligomer a central region that forms a substrate for RNase is flanked by hybridizing “arms” comprised of modified nucleotides that do not form substrates for RNase H. Alternatively, the substrate for RNase H that forms the “gap” can be on the 5′ or 3′ side of the oligomer (B. P. Monia, et al., J Biol Chem 268, 14514-22 (1993)). The “arms” which do not form substrates for RNase H have three relevant properties. First, they hybridize to the target providing the necessary duplex affinity to achieve antisense inhibition. Second, as discussed above, they reduce the number of phosphorothioate DNA linkages in the oligomer, thus reducing non-specific effects. Third, they limit the region that forms a substrate form RNase H, thus adding to the target specificity of the oligomer.

Several methods have been used to synthesize regions of chimeric oligomers which are not substrates for RNase H. For example, Dagle et al. synthesized chimeric oligomers with methylphosphonate and phosphoramidate linkages in the arms (Dagle, J. M., Walder, J. A. & Weeks, D. L. Nucleic Acids Res 18, 4751-7 (1990): Agrawal, S., Mayrand, S. H., Zamecnik, P. C. & Pederson, T. Proc Natl Acad Sci USA 87, 1401-5 (1990). While these compounds functioned well in buffer systems and Xenopus oocytes, the arms decreased the hybrid affinity. This decrease in affinity dramatically reduces the activity of oligomers in mammalian cell culture. Also, these neutral and/or other neutral or radically modified backbone chemistries are often difficult and expensive to synthesize. 2′ modified sugars (e.g., —O-alkyl and fluoro and other 2′ modifications) have excellent hybrid affinity, and thus are well suited for use in the “arms” of chimeric oligomers. In an earlier patent application by Monia (WO 94/08003 FIG. 15), oligomers are described that have 2′-O-methyl hybridizing “arms” without phosphorothioates in the “arms”. While Monia shows that these oligomers may function in some cases (WO 94/08003, see, e.g., FIG. 15), oligomers of this type have reduced activity in cellular systems. This may be due to exonuclease degradation of the 2′-O-methyl phosphodiester linkages.

In order to maximize therapeutic activity of antisense oligomers, it would be of great benefit to improve upon the prior art oligomers by optimizing the affinity of the oligomers for their target molecules, increasing the stability of the oligomers, decreasing the toxicity of the oligomers for cells and enhancing uptake of the oligomers by cells.

### SUMMARY OF THE INVENTION

The instant invention is based, at least in part, on the discovery that modifications to the prior art antisense oligomers result in improved properties. In addition, improved methods for facilitating uptake of oligomers have been developed. The invention improves the prior art antisense oligomers, inter alia, by increasing the affinity of the oligomers for their target molecules, increasing the resistance of the oligomers to nucleases, decreasing their toxicity, and optimizing uptake of the oligomers by cells.

Accordingly, the invention provides optimized antisense oligomer compositions and methods for making and using both in in vitro systems and therapeutically.

In one aspect, the invention features an oligomer comprising: an RNase H activating region and at least one nonactivating region, wherein the nonactivating region of the oligomer comprises at least one nucleomonomer having a 2′ OH propargyl group, said oligomer being sufficiently stabilized against nucleases.

In one embodiment, the oligomer further comprises 5′ and 3′ termini which are stabilized against exonucleases. In another embodiment, the oligomer is about 15-40 nucleomonomers in length.

In one aspect, the invention features chimeric antisense oligomers comprising a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 5′ terminal nucleomonomer is attached to an RNase H-activating region of between about three and ten contiguous phosphorothioate-linked nucleomonomers comprising deoxyribose, and wherein the 3′ terminus of said oligonucleotide is selected from the group consisting of: an inverted nucleomonomer, a contiguous stretch of about one to three phosphorothioate 2′-modified nucleomonomers, a biotin group, and a P-alkyloxyphosphotriester linked nucleomonomer, other modified nucleotide resistant to exonucleases, or non-nucleotide exonuclease blocking group, said oligomer having at least one nucleomonomer comprising a 2′ OH propargyl group.

In another aspect, a chimeric antisense oligomer comprises a 5′ terminus; a 3′ terminus; and 5′→3′ linked nucleomonomers independently selected from the group consisting of: 2′-modified phosphodiester linked nucleomonomers, and 2′-modified P-alkyloxyphosphotriester linked nucleomonomers; and wherein said 3′ terminal nucleomonomer is attached to an RNase H-activating region of between about three and ten contiguous phosphorothioate-linked nucleomonomers comprising deoxyribose, and wherein the 5′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester nucleomonomer, said oligomer having at least one nucleomonomer comprising a 2′ OH propargyl group.

In one aspect, a chimeric oligomer comprises: a 5′ terminus and a 3′ terminus, an RNase H activating region, and at least one nonactivating region, wherein a nonactivating region comprises at least one unmodified RNA ribonucleotide selected from the group consisting of: adenosine and guanine, said oligomer being sufficiently stabilized against nucleases.

In yet another aspect, a chimeric oligomer comprises: a 5′ terminus and a 3′ terminus, an RNase H activating region, and at least one nonactivating region, wherein a nonactivating region comprises a stretch between about 5 and about 10 of contiguous unmodified RNA ribonucleotides selected from the group consisting of: adenosine and guanine, said oligomer being sufficiently stabilized against nucleases.

In still another aspect, a chimeric antisense oligomer comprises a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 5′ terminal nucleomonomer is attached to an RNase H-activating region of between about three and ten contiguous phosphorothioate-linked nucleomonomers comprising deoxyribose, and wherein the 3′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester linked nucleomonomer said oligomer comprising a stretch of contiguous unmodified RNA nucleomonomers selected from the group consisting of: adenosine and guanine, said oligomer being sufficiently stabilized against nucleases.

In a further aspect, the invention features chimeric antisense oligomers comprising: a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 3′ terminal nucleomonomer is attached to an RNase H-activating region of between about three and ten contiguous phosphorothioate-linked nucleomonomers comprising deoxyribose, and wherein the 5′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester linked nucleomonomer said oligomer comprising a stretch of contiguous unmodified RNA nucleomonomers selected from the group consisting of: adenosine and guanine, said oligomer being sufficiently stabilized against nucleases.

In another aspect, the invention features an oligomer comprising: an RNase H activating region, at least one nonactivating region, and at least one affinity enhancing agent, wherein said affinity enhancing agent is not positioned adjacent to an RNase H activating region, said oligomer being sufficiently stabilized against nucleases.

In yet a further aspect, the invention features a chimeric antisense oligomer comprising a 5′ terminus; a 3′ terminus; and 5′-3′ linked nucleomonomers independently selected from the group consisting of 2′-modified phosphodiester linked nucleomonomers, and 2′-modified P-alkyloxyphosphotriester linked nucleomonomers; and wherein said 5′ terminal nucleomonomer is attached to an RNase H-activating region of between about three and ten contiguous phosphorothioate-linked nucleomonomers comprising deoxyribose, and wherein the 3′ terminus of said oligonucleotide is selected from the group consisting of: an inverted nucleomonomer, a contiguous stretch of one to three phosphorothioate linked 2′-modified nucleomonomers, a biotin group, and a P-alkyloxyphosphotriester linked nucleomonomer, said oligomer comprising at least one affinity enhancing agent, wherein said affinity enhancing agent is not positioned adjacent to an RNase H activating region.

In still another aspect, the invention provides a chimeric antisense oligomer comprising a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 3′ terminal nucleomonomer is attached to an RNase H-activating region of between about three and ten contiguous phosphorothioate-linked nucleomonomers comprising deoxyribose, and wherein the 5′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester linked nucleomonomer, said oligomer comprising at least one affinity enhancing agent, wherein said affinity enhancing agent is not positioned adjacent to an RNase H activating region.

In a further aspect, the invention provides compositions for inhibiting the expression of a protein in a cell comprising: an oligomer and a transporting peptide, wherein said transporting peptide is covalently attached to said oligomer. In one embodiment, the transporting peptide comprises a peptide selected from the group consisting of: an active portion of the antennapedia protein, an active portion of the transportan protein, and an active portion of the HIV TAT protein.

In another aspect, the invention provides a method for inhibiting the expression of a protein in a cell comprising contacting a cell with an oligomer. In one embodiment, the invention provides a method for delivering an oligomer to a cell comprising contacting the cell with a mixture comprising said oligomer and a cationic lipid for at least about three days.

### DRAWINGS

FIG. 1 illustrates the inhibition of luciferase activity by oligomers comprising propargyl modified nucleomonomers.

### DETAILED DESCRIPTION OF THE INVENTION

The instant invention advances the prior art by providing optimized antisense oligomer compositions for use in techniques and therapies and by providing methods of making and using the improved antisense oligomer compositions.

The term “oligomer” includes two or more nucleomonomers covalently coupled to each other by linkages or substitute linkages. An oligomer may comprise, for example, between a few (e.g. 7, 10, 12, 15) or a few hundred (e.g., 100 or 200) nucleomonomers. For example, an oligomer of the invention preferably comprises between about 10 and about 50 nucleomonomers, between about 15 and about 40, or between about 20 and about 30 nucleomonomers. More preferably, an oligomer comprises about 25 nucleomonomers. Oligomers may comprise, for example, oligonucleotides, oligonucleosides, polydeoxyribonucleotides (containing 2′-deoxy-D-ribose) or modified forms thereof, e.g., DNA, polyribonucleotides (containing D-ribose or modified forms thereof), RNA, or any other type of polynucleotide which is an N-glycoside or C-glycoside of a purine or pyrimidine base, or modified purine or pyrimidine base. The term oligomer includes compositions in which adjacent nucleomonomers are linked via phosphorothioate, amide and other linkages (e.g., Neilsen, P. E., et al. 1991. Science. 254:1497).

Oligomers comprise one or more regions which are complementary too and can bind to a target nucleic acid sequence, e.g., by Watson/Crick or Hoogsteen binding. Preferably, oligomers of the invention are substantially complementary to a target RNA sequence. By substantially complementary it is meant that no loops greater than about 8 nucleotides are formed by areas of non-complementarity between the oligomer and the target. In a preferred embodiment, the antisense oligomers of the invention are complementary to a target RNA sequence over at least about 80% of the length of the oligomer. In a more preferred embodiment, antisense oligomers of the invention are complementary to a target RNA sequence over at least about 90-95% of the length of the oligomer. In a more particularly preferred embodiment, antisense oligomers of the invention are complementary to a target RNA sequence over the entire of the length of the oligomer. The ability of an oligomer to bind to a target sequence is primarily a function of the bases in the oligomer. Accordingly, elements ordinarily found in oligomers, such as the furanose ring and/or the phosphodiester linkage can be replaced with any suitable functionally equivalent element. The term “oligomer” includes any structure that serves as a scaffold or support for the bases of the oligomer, where the scaffold permits binding to the target nucleic acid molecule in a sequence-dependent manner.

The term “nucleomonomer” includes bases covalently linked to a second moiety. Nucleomonomers include, for example, nucleosides and nucleotides. Nucleomonomers can be linked to form oligomers that bind to target nucleic acid sequences in a sequence specific manner. The term “second moiety” as used herein includes substituted and unsubstituted cycloalkyl moieties, e.g. cyclohexyl or cyclopentyl moieties, and substituted and unsubstituted heterocyclic moeities, e.g. 6-member morpholino moeities or, preferably, sugar moieties. Sugar moieties include natural sugars, e.g. monosaccharides (such as pentoses, e.g. ribose), modified sugars and sugar analogs. Possible modifications include, for example, replacement of one or more of the hydroxyl groups with a halogen, a heteroatom, an aliphatic group, or the functionalization of the group as an ether, an amine, a thiol, or the like. For example, modified sugars include D-ribose, 2′-O-alkyl, 2′-amino 2′-S-alkyl, 2′halo, 2′-O-methyl, 2′-fluoro, 2′-methyoxy, 2′-ethyoxy, 2′-methoxyethoxy, 2′-allyloxy (—OCH2CH═CH2), 2′-propargyl, 2′ propyl, ethynyl, ethenyl, propenyl, and cyano and the like. In one embodiment, the sugar moiety can be a hexose and incorporated into an oligomer as described (Augustyns, K., et al., Nucl. Acids. Res. 1992. 18:4711). Exemplary nucleomonomers can be found, e.g., in U.S. Pat. No. 5,849,902.

The term “base” includes the known purine and pyrimidine heterocyclic bases, deazapurines, and analogs (including heterocycl substituted analogs, e.g. aminoethyoxy phenoxazine), derivatives (e.g. 1-alkenyl-, 1-alkynyl-, heteroaromatic- and 1-alkynyl derivatives) and tautomers thereof. Examples of purines include adenine, guanine, inosine, diaminopurine, and xanthine and analogs (e.g., 8-oxo-N⁶methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids and/or amino acid analogs which may comprise free carboxyl groups, free amino groups, or protecting groups. Suitable protecting groups are well known in the art (see: T. W. Greene, “Protective Groups in Organic Synthesis”, Wiley, New York, 1981; J. F. W. McOmie (ed.), “Protective Groups in Organic Chemistry”, Plenum, N.Y., 1973).

The term “nucleotide” includes nucleosides which further comprise a phosphate group or a phosphate analog.

As used herein, the term “linkage” includes a naturally occurring, unmodified phosphodiester moiety (-O-P(O)(O)-O-) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., such as phosphorothioate, phosphorodithioate, and P-ethyoxyphosphodiester, p-ethoxyphosphodiester, p-alkyloxyphosphotriester, methylphosphnate, and nonphosphorus containing linkages, e.g., such as acetals and amides. Such substitute linkages are known in the art (e.g., Bjergarde et al. 1991. Nucleic Acids Res. 19:5843; Caruthers et al. 1991. Nucleosides Nucleotides. 10:47).

Oligomers of the invention comprise 3′ and 5′ termini. The 3′ and 5′ termini of an oligomer can be substantially protected from nucleases e.g., by modifying the 3′ and/or 5′ linkages (e.g., U.S. Pat. No. 5,849,902 and WO 98/13526.). For example, oligomers can be made resistant by the inclusion of a “blocking group.” The term “blocking group” as used herein refers to substituents (e.g., other than OH groups) that can be attached to oligomers or nucleomonomers, either as protecting groups or coupling groups for synthesis (e.g., hydrogen phosphonate, phosphoramidite, or PO₃⁻²).

“Blocking groups” also include “end blocking groups” or “exonuclease blocking groups” which protect the 5′ and 3′ termini of the oligomer, including modified nucleotides and non-nucleotide exonuclease resistant structures. Exemplary end-blocking groups include cap structures (e.g., a 7-methylguanosine cap), inverted nucleomonomers, e.g., with 3′-3′ and/or 5′-5′ end inversions (see e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl is esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages can be modified linkages. Optionally, the 5′ terminal hydroxy moiety can be esterified with a phosphorus containing moiety, e.g., phosphate, phosphorothioate, or P-ethoxyphosphate.

The term “chimeric oligomer” includes oligomers which comprise different component parts or regions which impart a desired quality to the oligomer. For example, specific regions of the oligomer (i.e., segments of the oligomer comprising at least one nucleomonomer) can provide stability against endonucleases, stability against exonucleases, complementarity with the target sequence, RNase H recruitment and activation, or the like. Regions may be multifunctional, e.g., providing more than one quality to the oligomer, e.g., complementarity and stability or RNase activation and complementarity. In addition, those of skill in the art will recognize that there may be more than one region imparting the same quality to one oligomer. The term “chimeric oligomer” includes oligomers having an RNA-like and a DNA-like region.

The language “RNase H activating region” includes a region of an oligomer, e.g. a chimeric oligomer, that is capable of recruiting RNase H to cleave the target RNA strand to which the oligomer is binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See e.g., U.S. Pat. No. 5,849,902). More preferably, the RNase H activating region comprises about nine deoxyribose containing nucleomonomers. Preferably, the contiguous nucleomonomers are linked by a substitute linkage, e.g., a phosphorothioate linkage.

The language “non-activating region” includes a region of an oligomer, e.g. a chimeric oligomer, that does not recruit or activate RNase H. Preferably, a non-activating region does not comprise phosphorothioate DNA. The oligomers of the invention comprise at least one non-activating region. A non-activating region can comprise between about 10 and about 30 nucleomonomers. The non-activating region can be stabilized against nucleases and/or can provide specificity for the target by being complementary to the target and forming hydrogen bonds with the target nucleic acid molecule, preferably mRNA molecule, which is to be bound by the oligomer.

Enhancing Affinity of Oligomers

In general, it is ideal for oligomers to have high affinity for their target nucleotide sequences; high affinity oligomers are more active. However, high affinity oligomers frequently display reduced specificity for their target, e.g., by binding to partially matched non-targeted sites. Such reduced specificity is undesirable in both research and clinical applications.

The oligomers of the instant invention solve this problem by providing increased affinity, while maintaining binding specificity for a target nucleotide sequence. This is accomplished by including in the oligomer an agent which increases the affinity of the oligomer for its target sequence.

The term “affinity enhancing agent” includes agents that increase the affinity of an oligomer for its target. Such agents include, e.g., intercalating agents and high affinity nucleomonomers. The agents may also impart other qualities to the oligomer, for example, increasing resistance to endonucleases and exonucleases.

In one embodiment, a high affinity nucleomonomer is incorporated into the oligomer. The language “high affinity nucleomonomer” as used herein includes modified bases or base analogs that bind to a complementary base in a target RNA molecule with higher affinity than an unmodified base, for example, by having more energetically favorable interactions with the complementary base, e.g., by forming more hydrogen bonds with the complementary base. For example, high affinity nucleomonomer analogs such as aminoethyoxy phenoxazine (also referred to as a G clamp), which forms four hydrogen bonds with guanine are included in the term “high affinity nucleomonomer.” A high affinity nucleomonomer is illustrated below.

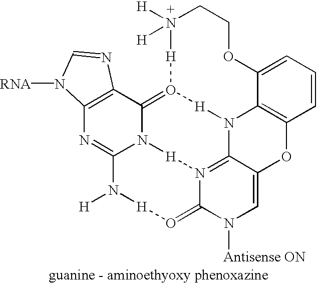

(See e.g., Flanagan et al. 1999. Proc. Natl. Acad. Sci. 96:3513).

Other exemplary high affinity nucleomonomers are known in the art and include 7, alkenyl, 7-alkynyl, 7-heteroaromatic- or 7-alkynyl-heteroaromatic-substituted bases or the like which can be substituted for adenosine or guanosine in oligomers (see e.g., U.S. Pat. No. 5,594,121). 7-substituted deazapurines have been found to impart enhanced binding properties to oligomers, i.e., by allowing them to bind with higher affinity to complementary target RNA molecules as compared to unmodified oligomers. High affinity nucleomonomers can be incorporated into the oligomers of the instant invention using standard techniques.

In another embodiment, an agent that increases the affinity of an oligomer for its target comprises an intercalating agent. As used herein the language “intercalating agent” includes agents which can bind to a DNA double helix. When covalently attached to an oligomer of the invention, an intercalating agent enhances the binding of the oligomer to its complementary genomic DNA target sequence. The intercalating agent may also increase resistance to endonucleases and exonucleases. Exemplary intercalating agents are taught by Helene and Thuong (1989. Genome 31:413), and include e.g., acridine derivatives (Lacoste et al. 1997. Nucleic Acids Research. 25:1991; Kukreti et al. 1997. Nucleic Acids Research. 25:4264); quinoline derivatives (Wilson et al. 1993. Biochemistry 32:10614); benzo[f]quino[3,4-b]quioxaline derivatives (Marchand et al. 1996. Biochemistry. 35:5022; Escude et al. 1998. Proc. Natl. Acad. Sci. 95:3591). Intercalating agents can be incorporated into an oligomer using any convenient linkage. For example, acridine or psoralen can be linked to the oligomer through any available —OH or —SH group, e.g., at the terminal 5′ position of the oligomer, the 2′ positions of sugar moieties, or an OH, NH2, COOH or SH incorporated into the 5-position of pyrimidines using standard methods.

In one embodiment, an oligomer comprises at least one agent that increases the affinity of an oligomer for its target. Preferably, oligomer comprises one agent that increase the affinity of an oligomer for its target.

In one embodiment, an agent that increases the affinity of an oligomer for its target is not positioned adjacent to an RNase activating regions of the oligomer, e.g., is positioned adjacent to a non-RNase activating region. Preferably, the agent that increases the affinity of an oligomer for its target is placed at a distance as far as possible from the RNase activating domain of the chimeric antisense oligomer such that the specificity of the chimeric antisense oligomer is not altered when compared with the specificity of a chimeric antisense oligomer which lacks the intercalating compound. In one embodiment, this can be accomplished by positioning the agent adjacent to a non-RNase activating region. The specificity of the oligomer can be tested by demonstrating that transcription of a non-target sequence, preferably a sequence which is structurally similar to the target (e.g., has some sequence homology or identity with the target sequence but which is not identical in sequence to the target) is not inhibited to a greater degree by an oligomer comprising an affinity enhancing agent than by an oligomer that does not comprise an affinity enhancing agent.

A variety of conformations of the subject oligomers are possible. For example, in one embodiment, a chimeric antisense oligomer is configured as depicted in the exemplary representation below (where A represents an RNase activating region of the oligomer, B represents a non-RNase H activating region (non-activating region) of the oligomer, B′ represents a non-activating region of the oligomer which is stable in the absence of an exonuclease blocking group (e.g., a 3′ exonuclease blocking group), G represents a high affinity nucleomonomer, and C represents an exonuclease blocking group):

A•B•G•C

In another embodiment, a chimeric antisense oligomer is configured as depicted in the exemplary representation below:

A•B′•G

In another embodiment, a chimeric antisense oligomer is configured as depicted in the exemplary representation below:

C•G•B•A•

In yet another embodiment, a chimeric antisense oligomer is configured as depicted in the exemplary representation below:

C•B•A•B•G•C

In yet another embodiment, a chimeric antisense oligomer is configured as depicted in the exemplary representation below:

C•G•B•A•B•C

Preferably, the affinity enhancing agent is positioned at a distance of at least about 5 to at least about 20 nucleomonomers from an RNase activating region. More preferably, the affinity enhancing agent is positioned at a distance of at least about 10 to at least about 15 nucleomonomers from an RNase activating region. In a particularly preferred embodiment, the affinity enhancing agent is positioned at a distance of at least about 12 nucleomonomers from an RNase activating region.

Enhancing Resistance of Oligomers to Nucleases Previous anti sense oligomers have made use of 2′-O-methyl groups for the hybridizing arms of chimeric oligomers (Inoue, H. et al. 1987. Nucleic Acids Res. 15:613 1). However, 2′-O-Methyl bases with unmodified phosphodiester linkages are degraded by exonucleases and, thus, are not optimal for inclusion in antisense oligomers (Shibahara, S., et al. 1989. Nucleic Acids Res. 17:239). Phosphorothioate linked 2′-O-methyl nucleomonomers can be incorporated into oligomers to enhance stability (Monia et al. 1993. J Biol. Chem. 268:14514). However, oligomers comprising fully phosphorothioate linked nucleomonomers may cause non-specific effects, including cell toxicity (Stein C. et al. 1989. Aids Res. Hum. Retrov. 5:639; Woolf, T., et al. 1990. Nucleic Acids Res. 18:1763; Wagner, R. W. 1995. Antisense Res. Dev. 5:113; Krieg, A., and Stein, C. 1995. Antisense Res Dev. 5:241). In addition, each incorporation of a phosphorothioate generates a chiral center and reduces the binding affinity for target mRNA by 1-1.5° C. (Dean and Griffey. 1997. Antisense and Nucleic Acid Drug Development. 7:229).

The instant oligomers improve upon the prior art oligomers by incorporating nucleomonomers having 2′-propargyl (i.e., CH₂—C≡CH) groups, e.g., nucleomonomers having 2′-propargyl groups attached to the second moiety of the nucleomonomer. Preferably, an oligomer comprises nucleomonomers having propargyl groups linked to the 2′ OH of a sugar moiety of a nucleomonomer. 2′-O-propargyl groups provide a surprising increase in stability over that imparted by 2′O-methyl groups and allow for a reduction in the number of phosphorothioate linkages in the oligomer. Oligomers containing 2′O-propargyl modified nucleomonomers can be synthesized using standard phosphoramidite protocols. The 2′ O-propargyl phosphoramidite (nucleomonomer) is commercially available (e.g., from ChemGenes, Waltham, Mass.) and can be incorporated into oligomers of the invention without further modification.

The synthesis of oligomers comprising 2′ O-propargyl modified nucleotides is described in Example 1. A propargyl-modified second moiety is illustrated in B below. In contrast to the ribose nucleotide shown in A, the 2′ O-propargyl modified nucleotide comprises a propargyl group in the 2′ position attached via an ester linkage.

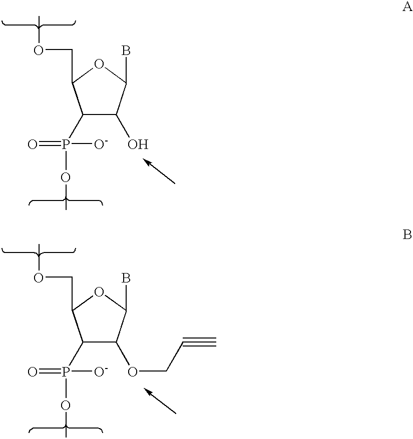

Propargyl groups are present in the non-activating region of the oligomers of the invention. In one embodiment, one nucleomonomer comprising a propargyl group is present in the non-activating region. In another embodiment more than one nucleomonomer comprising a propargyl group is present in the non-activating region of an oligomer. In one embodiment, an oligomer comprises more than one adjacent nucleomonomer comprising propargyl, i.e., providing a contiguous stretch of propargyl-modified nucleomonomers. In another embodiment, propargyl-modified nucleomonomers are not present in a contiguous stretch, e.g., are adjacent to nucleomonomers that do not comprise propargyl groups.

An exemplary oligomer comprising propargyl groups is illustrated by the construct below (where A represents an RNase activating region and P represents a nonactivating region containing nucleomonomers comprising a propargyl modification, and C represents an exonuclease blocking group).

5′ A•P•C

In one embodiment, adjacent nucleomonomers comprising propargyl groups are linked via modified linkages. In another embodiment, adjacent nucleomonomers comprising 2′-O propargyl groups are linked via phosphodiester linkages.

Modification of Oligomers to Minimize Toxicity

Many of the modifications which are made to oligomers in an effort to enhance nuclease resistance increase the toxicity of oligomers. In one embodiment, the instant invention improves upon the prior art oligomers by providing oligomers which comprise at least one unmodified ribonucleotide. In one embodiment of the invention, one or more nucleomonomers of an oligomer are present as unmodified RNA nucleomonomers. Unmodified RNA is non-toxic to cells, but was thought to be too unstable for use in antisense oligomers. However, the instant invention provides several means by which unmodified RNA can be incorporated into antisense constructs. In addition to being nontoxic, unmodified nucleomonomer precursors are less expensive to make than are modified RNA precursors.

For example, unmodified RNA containing the ribonucleotides cytidine (C) and/or uradine (U) is rapidly degraded in serum, RNA devoid of C's and U's has been found to be stable to most RNases (Heidenreich, et al. J Biol Chem 269,2131-8 (1994). Accordingly, in one embodiment, an oligomer is designed to comprise a region devoid of C's and/or U's, i.e., a region rich in the ribonucleotides adenosine (A) and/or guanosine (G). In cases where activation of RNase H is desired, a region of hosphorothioate DNA is included in the oligomer.

For example, target sites rich an C's and U's can be identified in a target RNA molecule. Preferably, target sites will comprise at least about 10 to at least about 12 contiguous C's and/or U's. Target sites having such a stretch of ribonucleotides can be identified in a mRNA molecule to be cleaved. Once the target sequence is selected, a chimeric antisense oligomer is configured. The unmodified RNA nucleomonomer(s) can be present at any position in a non-activating region of the oligomer, for example, as depicted in the exemplary representations below (where A represents an RNase activating region of the oligomer, B represents a region of the oligomer which does not activate RNase H and is rich in A's and/or G's which complement the sequence of the target RNA molecule, and C represents an exonuclease block):

A•B•C

C•B•A

(B=e.g., AGAGAG; SEQ ID NO: 1)

When more than one unmodified ribonucleotide is present in an oligomer, the unmodified ribonucleotides need not be present in a contiguous stretch. For example, a non-activating region of an oligomer can comprise unmodified RNA and modified RNA nucleomonomers, e.g., in the exemplary representations above, region B in addition to comprising unmodified RNA nucleomonomers, can comprise at least one 2′ modified C and/or U and/or one 2′ modified A or G.

In preferred embodiments, the oligomers preferably comprise an end-blocking group on the 3′ and/or 5′ terminus of the oligomer (see e.g., U.S. Pat. No. 5,849,902). In such an end-blocked oligomer, all of the A's and G's present in the nonactivating region of the oligomer can be replaced with unmodified RNA nucleomonomers. Another exemplary oligomer comprising unmodified RNA is illustrated by:

T(ps)T(ps)G(ps)C(ps)C(ps)C(ps)A(ps)C(ps)A(ps)CCgaCggCgCCCaCCa(ps)3′ end block (SEQ ID NO: 2).

(where upper case nucleomonomers are DNA, lower case nucleomonomers are RNA, underlined uppercase nucleomonomers are 2′O-methyl RNA, the 3′ block is an inverted nucleomonomer, e.g., an inverted thymine (T). Phosphorothioate linkages are illustrated by “(ps),” unmarked linkages are phosphodiester linkages.

Uptake of Oligomers by Cells

Oligomers need to be delivered to, e.g., contacted with and taken up by one or more cells. The term “cells” refers to prokaryotic and eukaryotic cells, preferably vertebrate cells, and, more preferably, mammalian cells. In a preferred embodiment, oligomers are contacted with human cells. Oligomers can be contacted with cells in vitro or in vivo. Oligomers are taken up by cells at a slow rate by endocytosis, but endocytosed oligomers are generally sequestered and not available for hybridization to target RNA. Cellular uptake can be facilitated by electroporation or calcium phosphate precipitation. However, these procedures are only useful for in vitro or ex vivo embodiments, are not convenient and, in some cases, are associated with cell toxicity.

Delivery of oligomers into cells can be enhanced by suitable art recognized methods including calcium phosphate, DMSO, glycerol or dextran, electroporation, or by transfection, e.g., using cationic, anionic, and/or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligomers can also be mediated by the use of viruses, polyamine or polycation conjugates using compounds such as polylysine, protamine, or N1, N12-bis (ethyl) spermine (see e.g., Bartzatt, R. et al. 1989. Biotechnol. Appl. Biochem. 11:133; Wagner E. et al. 1992. Proc. Natl. Acad. Sci. 88:4255) In one embodiment, oligomers can be derivitized or chemically modified to facilitate cellular uptake. For example, covalent linkage of a cholesterol moiety to an oligomer can improve cellular uptake by 5- to 10-fold which in turn improves DNA binding by about 10-fold (Boutorin et al., 1989, FEBS Letters 254:129-132). Similarly, derivatization of oligomers with poly-L-lysine can aid oligomer uptake by cells (Schell, 1974, Biochem. Biophys. Acta 340:323, and Lemaitre et al., 1987, Proc. Natl. Acad. Sci. USA 84:648). Certain protein carriers can also facilitate cellular uptake of oligomers, including, for example, serum albumin, nuclear proteins possessing signals for transport to the nucleus, and viral or bacterial proteins capable of cell membrane penetration. Therefore, protein carriers are useful when associated with or linked to the oligomers. Accordingly, the present invention contemplates derivatization of oligomers with groups capable of facilitating cellular uptake, including hydrocarbons and non-polar groups, cholesterol, poly-L-lysine and proteins, as well as other aryl or steroid groups and polycations having analogous beneficial effects, such as phenyl or naphthyl groups, quinoline, anthracene or phenanthracene groups, fatty acids, fatty alcohols and sesquiterpenes, diterpenes and steroids.

In another embodiment, an oligomer may be associated with a carrier or vehicle, e.g., liposomes or micelles, although other carriers could be used, as would be appreciated by one skilled in the art. Such carriers are used to facilitate the cellular uptake and/or targeting of the oligomer, and/or improve the oligomer's pharmacokinetic and/or toxicologic properties. For example, the oligomers of the present invention may also be administered encapsulated in liposomes, pharmaceutical compositions wherein the active ingredient is contained either dispersed or variously present in corpuscles consisting of aqueous concentric layers adherent to lipidic layers. The oligomers, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, and/or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a liposome delivery vehicle originally designed as a research tool, such as Lipofectin, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

Cationic lipids can also be used to deliver oligomers to cells. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl—, Br—, I—, F—, acetate, trifluoroacetate, sulfate, nitrite, and nitrate.

Cationic lipids have been used in the art to deliver oligomers to cells (See e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligomers can be used in connection with the claimed methods. In addition to those listed supra, other lipid compositions are also known in the art and include, e.g., those taught in U.S. Pat. No. 4,235,871; U.S. Pat. No. 4,501,728; 4,837,028; 4,737,323. In one embodiment lipid compositions can further comprise agents, e.g., viral proteins to enhance lipid-mediated transfections of oligomers (Kamata et al. 1994. Nucl. Acids. Res. 22:536). In another embodiment, oligomers are contacted with cells as part of a composition comprising an oligomer, a peptide, and a lipid as taught, e.g., in U.S. Pat. No. 5,736,392. Improved lipids have also been described which are serum resistant (Lewis et al. 1996. Proc. Natl. Acad. Sci 93:3176)

In another embodiment N-substituted glycine oligomers (peptoids) can be used to optimize uptake of oligomers. Peptoids have been used to create cationic lipid-like compounds for transfection (Murphy et al. 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R.N., et al. 1992. Int. J Peptide Protein Res. 40:497). Combinations of cationic lipids and peptoids, liptoids, can also be used to optimize uptake of the subject oligomers (Hunag et al. 1998. Chemistry and Biology. 5:345). Liptoids can be synthesized by elaborating peptoid oligomers and coupling the amino terminal submonomer to a lipid via its amino group (Hunag et al. 1998. Chemistry and Biology. 5:345).

It is known in the art that positively charged amino acids can be used for creating highly active cation lipids (Lewis et al. 1996. Proc. Natl. Acad. Sci. U.S.A. 93:3176). In one embodiment, a composition for delivering oligomers of the invention comprises a number of arginine, lysine, histadine and/or ornithine residues linked to a lipophilic moiety (see e.g., U.S. Pat. No. 5,777,153). In another, a composition for delivering oligomers of the invention comprises a peptide having from between about one to about four basic residues. These basic residues can be located, e.g., on the amino terminal, c-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used. For example, a peptide such as (N-term) His-Ile-Trp-Leu-Ile-Tyr-Leu-Trp-Ile-Val-(C-term) (SEQ ID NO: 3) could be used. In one embodiment such a composition can be mixed with the fusogenic lipid DOPE as is well known in the art.

In one embodiment, the cells to be contacted with an antisense construct are contacted with a mixture comprising the antisense construct and a mixture comprising a lipid, e.g., one of the lipids or lipid compositions described supra for between about 1 and about five days. In one embodiment, the cells are contacted with a mixture comprising a lipid and the antisense oligomer for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days. In a preferred embodiment, a mixture comprising a lipid is left in contact with the cells for at least about three days. Surprisingly, given the low toxicity of the instant oligomers, such prolonged incubation periods are possible.

For example, in one embdoiment, an oligomer having the configuration C•B•A•B•C; A•B•C; or A•B•A (where A represents an RNase activating region, B represents a non-activating region, and C represents an exonuclease blocking group), can be contacted with cells in the presence of a lipid such as cytofectin CS or GSV(available from Glen Research; Sterling, Va.), GS3815, GS2888 for prolonged incubation periods as described herein.

In one embodiment the incubation of the cells with the mixture comprising a lipid and the antisense construct does not reduce the viability of the cells. Preferably, after the transfection period the cells are substantially viable. In one embodiment, after transfection, the cells are between at least about 70 and at least about 100 percent viable. In another embodiment, the cells are between at least about 80 and at least about 95% viable. In yet another embodiment, the cells are between at least about 85% and at least about 90% viable. Preferably, the cells are no less viable at the end of the incubation period with the mixture comprising the antisense construct and the lipid than similarly treated cells that are incubated with the same mixture for a period of only about 24 hours or less. Preferably, the prolonged transfection period is used to deliver the oligomers of the instant invention to a cell.

In one embodiment, oligomers are modified by attaching a peptide sequence that transports the oligomer into a cell, referred to herein as a “transporting peptide.” In one embodiment, the composition includes an oligomer which is complementary to a target nucleic acid molecule encoding the protein, and a covalently attached transporting peptide.

The language “transporting peptide” includes an amino acid sequence that facilitates the transport of an oligomer into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

For example, in one embodiment, the transporting peptide comprises an amino acid sequence derived from the antennapedia protein. Preferably, the peptide comprises amino acids 43-58 of the antennapedia protein (Arg-Gln-Ile-Lys-Ile-Trp-Phe-Gln-Asn-Arg-Arg-Met-Lys-Trp-Lys-Lys) (SEQ ID NO: 4) or a portion or variant thereof that facilitates transport of an oligomer into a cell (see, e.g., WO 91/1898; Derossi et al. 1998. Trends Cell Biol. 8:84). Exemplary variants are shown in Derossi et al., supra.

In one embodiment, the transporting peptide comprises an amino acid sequence derived from the transportan, galanin (1-12)-Lys-mastoparan (1-14) amide, protein. (Pooga et al. 1998. Nature Biotechnology 16:857). Preferably, the peptide comprises the amino acids of the transportan protein shown in the sequence GWTLNSAGYLLGKINLKALAALAKKIL (SEQ ID NO: 5) or a portion or variant thereof that facilitates transport of an oligomer into a cell.

In one embodiment, the transporting peptide comprises an amino acid sequence derived from the HIV TAT protein. Preferably, the peptide comprises amino acids 37-72 of the HIV TAT protein, e.g., shown in the sequence C(Acm)FITKALGISYGRKKRRQRRRPPQC (SEQ ID NO: 6) (TAT 37-60; where C(Acm) is Cys-acetamidomethyl) or a portion or variant thereof, e.g., C(Acm)GRKKRRQRRRPPQC (SEQ ID NO: 7) (TAT 48-40) or C(Acm)LGISYGRKKRRQRRPPQC (SEQ ID NO: 8) (TAT 43-60) that facilitates transport of an oligomer into a cell (Vives et al. 1997. J. Biol. Chem. 272:16010). In another embodiment the peptide (G)CFITKALGISYGRKKRRQRRRPPQGSQTHQVSLSKQ (SEQ ID NO: 9)can be used.

Portions or variants of transporting peptides can be readily tested to determine whether they are equivalent to these peptide portions by comparing their activity to the activity of the native peptide, e.g., their ability to transport fluorescently labeled oligomers to cells. Fragments or variants that retain the ability of the native transporting peptide to transport an oligomer into a cell are functionally equivalent and can be substituted for the native peptides.

Oligomers can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J Biol. Chem. 272:16010). For example, in one embodiment, oligomers bearing an activated thiol group are linked via that thiol group to a cysteine present in a transport peptide (e.g., to the cysteine present in the b turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J Cell Biol. 128:919). In another embodiment, a Boc-Cys-(Npys)OH group can be coupled to the transport peptide as the last (N terminal) amino acid and an oligomer bearing an SH group can be coupled to the peptide (Troy et al. 1996. J. Neurosci. 16:253). In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₁-C₂₀ alkenyl chains, C₁-C₂₀ alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see e.g., Smith et al. Biochem J 1991. 276: 417-2).

In one embodiment, oligomers of the invention are synthesized as molecular conjugates which utilize receptor-mediated endocytotic mechanisms for delivering genes into cells (See e.g., Bunnell et al. 1992. Somatic Cell and Molecular Genetics. 18:559 and the references cited therein).

Assays of Oligomer Stability

The oligomers of the invention are stabilized, e.g., substantially resistant to endonuclease and exonuclease degradation. An oligomer is defined as being substantially resistant to nucleases when it is at least about 3-fold more resistant to attack by an endogenous cellular nuclease, and is highly nuclease resistant when it is at least about 6-fold more resistant than a corresponding oligomer comprised of unmodified DNA or RNA or, in the case of the instant oligomers designed to comprise AG rich unmodified RNA, when compared to oligomers comprising unmodified RNA not selected to be AG rich. This can be demonstrated by showing that the oligomers of the invention are substantially resist nucleases using techniques which are known in the art.

One way in which substantial stability can be demonstrated is showing that the oligomers of the invention function when delivered to a cell, e.g., that they reduce transcription of target RNA molecules, e.g., by measuring protein levels or by measuring cleavage of mRNA. Assays which measure the stability of target RNA can be performed at about 24 hours post-transfection (e.g., using Northern blot techniques, RNase Protection Assays, or QC-PCR assays as known in the art. Alternatively, levels of the target protein can be measured. Preferably, in addition to testing the RNA and/or protein levels of interest, the RNA and/or protein levels of a control, non-targeted gene will be measured (e.g., actin, or preferably a control with sequence similarity to the target) as a specificity control. Preferably, RNA and/or protein measurements will be made using any art-recognized technique. Preferably, measurements will be made beginning at about 16-24 hours post transfection. (M. Y. Chiang, et al. 1991. J. Biol Chem. 266:18162-71; T. Fisher, et al. 1993. Nucleic Acids Research. 21 3857.

Oligomer Synthesis

Oligomers of the invention can be synthesized by any methods known in the art, e.g., using enzymatic synthesis and chemical synthesis.

Preferably, chemical synthesis is used. Chemical synthesis of linear oligomers is well known in the art and can be achieved by solution or solid phase techniques. Preferably, synthesis is by solid phase methods. Oligomers can be made by any of several different synthetic procedures including the phosphoramidite, phosphite triester, H-phosphonate and phosphotriester methods, typically by automated synthesis methods. Oligomer synthesis protocols are well known in the art and can be found, e.g., in U.S. Pat. No. 5,830,653; WO 98/13526; Stec et al. 1984. J Am. Chem. Soc. 106:6077; Stec et al. 1985. J Org. Chem. 50:3908; Stec et al. J. Chromatog. 1985. 326:263; LaPlanche et al. 1986. Nuc. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; U.S. Pat. No. 5,264,423).

The synthesis method selected can depend on the length of the desired oligomer and such choice is within the skill of the ordinary artisan. For example, the phosphoramidite and phosphite triester method produce oligomers having 175 or more nucleotides while the H-phosphonate method works well for oligomers of less than 100 nucleotides. If modified bases are incorporated into the oligomer, and particularly if modified phosphodiester linkages are used, then the synthetic procedures are altered as needed according to known procedures. In this regard, Uhlmann et al. (1990, Chemical Reviews 90:543-584) provide references and outline procedures for making oligomers with modified bases and modified phosphodiester linkages. Other exemplary methods for making oligomers are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligomers of defined sequence can be purchased commercially.

The oligomers may be purified by polyacrylamide gel electrophoresis, or by any of a number of chromatographic methods, including gel chromatography and high pressure liquid chromatography. To confirm a nucleotide sequence, oligomers may be subjected to DNA sequencing by any of the known procedures, including Maxam and Gilbert sequencing, Sanger sequencing, capillary electrophoresis sequencing the wandering spot sequencing procedure or by using selective chemical degradation of oligomers bound to Hybond paper. Sequences of short oligomers can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligomers.

The quality of oligomers synthesized can be verified by testing the oligomer by capillary electrophoresis and denaturing strong anion HPLC (SAX-HPLC) using, e.g., the method of Bergot and Egan. 1992. J. Chrom. 599:35.

It will be understood that the oligomers of the invention can be synthesized to comprise one or more of the disclosed improvements. For example, in one embodiment, an oligomer of the invention comprises a nucleomonomer containing a propargyl group. In another embodiment, an oligomer of the invention comprises a nucleomonomer containing an affinity enhancing agent. In another exemplary embodiment, an oligomer of the invention comprises unmodified RNA nucleomonomers. In one embodiment, an oligomer of the invention comprises at least two of the above improvements. In one embodiment, an oligomer of the invention comprises at least three of the above improvements. One of skill in the art will recognize that given the teachings of the specification, multiple variations and combinations of these improved oligomers can be made.

Uses of Oligomers

The oligomers of the invention can be used in a variety of in vitro and in vitro situations to specifically degrade a target mRNA molecule. The instant methods and compositions are suitable for both in vitro and in vivo use.

In one embodiment, the oligomers of the invention can be used to inhibit gene function in vitro in a method for identifying the functions of genes. The transcription genes that are identified, but for which no function has yet been shown can be inhibited to determine how the phenotype of a cell is changed when the gene is not transcribed. Such methods are useful for the validation of target genes for clinical treatment with antisense oligomers or with other therapies.

In one embodiment, in vitro treatment of cells with oligomers can be used for ex vivo therapy of cells removed from a subject (e.g., for treatment of leukemia or viral infection) or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to study gene regulation and protein synthesis or to evaluate improvements made to oligomers designed to modulate gene expression and/or protein synthesis. In vivo treatment of cells can be useful in certain clinical settings where it is desirable to inhibit the expression of a protein. There are numerous medical conditions for which antisense therapy is reported to be suitable (see e.g., U.S. Pat. No. 5,830,653) as well as respiratory syncytial virus infection (WO 95/22553) influenza virus (WO 94/23028), and malignancies (WO 94/08003). Other examples of clinical uses of antisense oligomers are reviewed, e.g., in Glaser. 1996. Genetic Engineering News 16:1. Exemplary targets for cleavage by antisense oligomers include e.g., protein kinase Ca, ICAM-1, c-raf kinase, p53, c-myb, and the bcr/abl fusion gene found in chronic myelogenous leukemia.

The optimal course of administration of the oligomers may vary depending upon the desired result or on the subject to be treated. As used herein “administration” refers to contacting cells with oligomers. The dosage of oligomers may be adjusted to optimally reduce expression of a protein translated from a target mRNA, e.g., as measured by a readout of RNA stability or by a therapeutic response, without undue experimentation. For example, expression of the protein encoded by the nucleic acid target can be measured to determine whether or dosage regimen needs to be adjusted accordingly. In addition, an increase or decrease in RNA and/or protein levels in a cell or produced by a cell can be measured using any art recognized technique. By determining whether transcription has been decreased, the effectiveness of the oligomer in inducing the cleavage of the target RNA can be determined.

As used herein, “pharmaceutically acceptable carrier” includes appropraite solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Oligomers may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligomers to specific cell types.

Moreover, the present invention provides for administering the subject oligomers with an osmotic pump providing continuous infusion of such oligomers, for example, as described in Rataiczak et al. (1992 Proc. Natl. Acad. Sci. USA 89:11823-11827). Such osmotic pumps are commercially available, e.g., from Alzet Inc. (Palo Alto, Calif.). Topical administration and parenteral administration in a cationic lipid carrier are preferred.

With respect to in vivo applications, the formulations of the present invention can be administered to a patient in a variety of forms adapted to the chosen route of administration, namely, parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation. Intravenous administration is preferred among the routes of parenteral administration.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran, optionally, the suspension may also contain stabilizers.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The described oligomers may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the oligomer to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the oligomer at the lymph node. The oligomer can be modified to diffuse into the cell, or the liposome can directly participate in the delivery of either the unmodified or modified oligomer into the cell.

The chosen method of delivery will result in entry into cells. Preferred delivery methods include liposomes (10-400 nm), hydrogels, controlled-release polymers, and other pharmaceutically applicable vehicles, and microinjection or electroporation (for ex vivo treatments).

The oligomers, especially in lipid formulations, can also be administered by coating a medical device, for example, a catheter, such as an angioplasty balloon catheter, with a cationic lipid formulation. Coating may be achieved, for example, by dipping the medical device into a lipid formulation or a mixture of a lipid formulation and a suitable solvent, for example, an aqueous-based buffer, an aqueous solvent, ethanol, methylene chloride, chloroform and the like. An amount of the formulation will naturally adhere to the surface of the device which is subsequently administered to a patient, as appropriate. Alternatively, a lyophilized mixture of a lipid formulation may be specifically bound to the surface of the device. Such binding techniques are described, for example, in K. Ishihara et al., Journal of Biomedical Materials Research, Vol. 27, pp. 1309-1314 (1993), the disclosures of which are incorporated herein by reference in their entirety.

The useful dosage to be administered and the particular mode of administration will vary depending upon such factors as the cell type, or for in vivo use, the age, weight and the particular animal and region thereof to be treated, the particular oligomer and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved. When lipids are used to deliver the oligomers, the amount of lipid compound that is administered can vary and generally depends upon the amount of oligomer agent being administered. For example, the weight ratio of lipid compound to oligomer agent is preferably from about 1:1 to about 15:1, with a weight ratio of about 5:1 to about 10:1 being more preferred. Generally, the amount of cationic lipid compound which is administered will vary from between about 0.1 milligram (mg) to about 1 gram (g). By way of general guidance, typically between about 0.1 mg and about 10 mg of the particular oligomer agent, and about 1 mg to about 100 mg of the lipid compositions, each per kilogram of patient body weight, is administered, although higher and lower amounts can be used.

The agents of the invention are administered to subjects or contacted with cells in a biologically compatible form suitable for pharmaceutical administration. By “biologically compatible form suitable for administration” is meant that the oligomer is administered in a form in which any toxic effects are outweighed by the therapeutic effects of the oligomer. In one embodiment, oligomers can be administered to subjects. The term subject is intended to include living organisms, e.g., prokaryotes and eukaryotes. Examples of subjects include mammals, e.g., humans, dogs, cats, mice, rats, and transgenic non-human animals.

Administration of an active amount of an oligomer of the present invention is defined as an amount effective, at dosages and for periods of time necessary to achieve the desired result. For example, an active amount of an oligomer may vary according to factors such as the type of cell, the oligomer used, and for in vivo uses the disease state, age, sex, and weight of the individual, and the ability of the oligomer to elicit a desired response in the individual. Establishment of therapeutic levels of oligomers within the cell is dependent upon the rates of uptake and efflux degradation. Decreasing the degree of degradation prolongs the intracellular half-life of the oligomer. Thus, chemically-modified oligomers, e.g., with modification of the phosphate backbone, may require different dosing.

The exact dosage of an oligomer and number of doses administered will depend upon the data generated experimentally and in clinical trials. Several factors such as the desired effect, the delivery vehicle, disease indication, and the route of administration, will affect the dosage. The expected in vivo dosage is between about 0.001-200 mg/kg of body weight/day. For example, the oligomers can be provided in a therapeutically effective amount of about 0.1 mg to about 100 mg per kg of body weight per day, and preferably of about 0.1 mg to about 10 mg per kg of body weight per day, to bind to a nucleic acid in accordance with the methods of this invention. Dosages can be readily determined by one of ordinary skill in the art and formulated into the subject pharmaceutical compositions. Preferably, the duration of treatment will extend at least through the course of the disease symptoms.

Dosage regima may be adjusted to provide the optimum therapeutic response. For example, the oligomer may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject oligomers, whether the oligomers are to be administered to cells or to subjects.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, NY (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The invention is further illustrated by the following examples, which should not be construed as further limiting. The contents of all references, pending patent applications and published patents, cited throughout this application are hereby expressly incorporated by reference.

### EXAMPLES

### Example 1

### Synthesis of Oligomers Comprising Propargyl Modifications

Chimeric oligomers containing 2′ O-propargyl phosphoramidite were synthesized using standard phosphoramidite protocols. The 2′O-propargyl phosphoramidite nucleomonomer was purchased commercially (e.g., from ChemGenes, Waltham, Mass.) and used without further modification. After the synthesis was complete, the oligomers were removed from the solid support and deprotected under standard conditions. The product was purified by Reversed-phase HPLC using a column composed of C 18 with a triethylammonium acetate/acetonitrile gradient. After purification, the oligomer was ethanol precipitated and then resuspended in 20 mM HEPES pH=8.0.

Configuration and Chemistries of Antisense Oligonucleotides

The following oligonucleotide configurations are shown in 5′ to 3′ orientation. The capital letter “X” represents deoxy ribonucleotides (DNA) while the lower case letter “x” represent nucleomonomers containing 2′ sugar modifications. The 2′ O-methyl and 2′ O-propargyl modified nucleomonomers are indicated by brackets. The 2′O-propargyl modified nucleomonomers are in bold. Phosphorothioate linkages between nucleotides are indicated by the symbol(ps). All other linkages are of the phosphodiester type. The three shown oligomers are 25 nucleotides in length and contain 3′ phosphorothioate linkages to a propyl group.

Control Oligomer
5′ X(ps)X(ps)X(ps)X(ps)X(ps)X(ps) X(ps)X(ps)X(ps) [xxxxxxxxxxxxxxxx]
(ps)(propyl) 3′
Propargyl #1
5′ X(ps)X(ps)X(ps)X(ps)X(ps)X(ps)X(ps)X(ps) X(ps) [x(ps)x(ps)x(ps)x(ps)x(ps)
(ps)x(ps)x(ps)x(ps)x(ps)x(ps)x(ps)x(ps)x(ps)x(ps)x](ps)(propyl) 3′
Propargyl #2
5′ X(ps)X(ps)X(ps)X(ps)X(ps)X(ps)X(ps)X(ps)X(ps) [xxxxxxxxxxxxxxx](ps)(propyl) 3′

Antisense Activity of Propargyl-Containing Oligomers

Oligomers containing the configuration and chemistries described above were designed to be antisense to a target sequence which has been spliced into the firefly luciferase messenger RNA (mRNA) sequence. Antisense activity results in cleavage of the target firefly luciferase mRNA, rapid degradation of the cleavage products and reduction in firefly luciferase activity. A control firefly luciferase mRNA does not contain the target sequence. Antisense oligomers were transfected into HeLa cells along with expression vectors for the target and control firefly luciferase mRNAs according to the protocol outlined below.

HeLa cells were grown in DMEM supplemented with 10% FBS, L-glutamine, penicillin and streptomycin. Cells were plated at 3.5×10⁵ cells/well in 24-well plates and incubated overnight. Lipofectin (Gibco/BRL, Gaithersberg, Md.) was diluted to 3.3 μg per milliliter in reduced serum medium (Opti-MEM, Gibco/BRL). Oligomers were added to the OptiMEM/Lipofectin mixture to a final concentration of 200 nanomolar from 100 μM concentrated stocks. The solution was mixed gently and complexes allowed to form for 15 minutes at room temperature. The normal growth medium was removed and the cells were rinsed once in OptiMEM. The Opti-MEM/Lipofectin/oligomer solution was then added to the cells and incubated for 4 hours (0.5 mls for one well of a 4 well plate). During this incubation a target transfection mixture was prepared by first diluting 3.3 μl of Lipofectin per ml of Opti-MEM and mixing. Two hundred nanograms of target firefly luciferase expression vector and 40 ng of internal control (renilla luciferase expression vector) were added per milliliter of Opti-MEM/Lipofectin mixture. The transfection mixture was mixed gently and allowed to complex for 15 minutes. A control experiment was also performed in which the firefly luciferase did not contain the target sequence. The oligonucleotide-containing media was removed from the cells and replaced with the ‘target’ and ‘control’ transfection mixtures (0.5 ml per well) and incubated for 2 hours. The second transfection mixture was removed and replaced with growth media and incubated for an additional 18 hours. The cells were then lysed in passive lysis buffer and luciferase activities in cell lysates were measured using the Dual luciferase Assay kit (Promega, Madison Wis.). Luminescence was detected using a 96 well luminometer (Packard, Meriden Ct). Firefly luciferase activity was normalized to internal control renilla luciferase activity. The data is expressed as the ratio of targeted firefly luciferase signal to non-targeted firefly luciferase signal.

Results

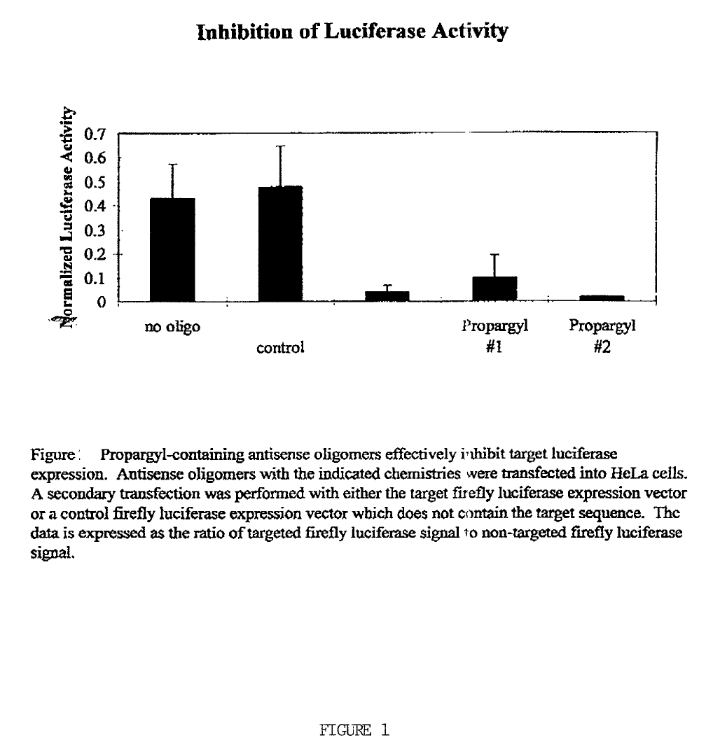

As shown in FIG. 1, the control antisense oligomer inhibits target luciferase activity by 91%. A second control oligomer that is not targeted to the luciferase mRNA has no effect. The propargyl containing oligomer (Propargyl #2, in which the propargyl modified nucleotides are linked with phosphodiester linkages) inhibits targeted luciferase activity by 96%. This result indicates that the incorporation of 2′O-propargyl modified nucleotides enhances the antisense activity of oligonucleotides.

### EXAMPLE 2

### Use of Cationic Lipids for Prolonged Periods of Time to Deliver Antisense Oligomers

Cells were treated with a mixture comprising between 50 nM-700 nM of an antisense oligomer and lipofectin (Gibco/BRL, Gaithersberg, Md.). This treatment has been found to result in 75-90% percent inhibition of expression of the target mRNA, depending on the target sequence chosen. Preferably, the oligomer is used at a concentration of about 200 nM. In these experiments, it was found that the inhibition persisted for 1-2 days after the transfection of the oligomer. In general, at lower cell confluence, the cationic lipid/oligomer complexes are more toxic to the cells. If cell confluence is too high, however, the uptake of the oligomer may be reduced.

For delivering oligomers to cells, lipid compositions, e.g., GSV or lipofectin, can be used as recommended by the manufacturer (e.g., Glen Research, Sterling, Va.). For example, in one step of contacting cells with an oligomer, about 3.3 μl or about 1.25 μl of GSV can be added to about 100 μl of medium, e.g., Opti-MEM®.

Preferably, a vessel used to contain GSV does not comprise polypropylene. In addition, when GSV is employed, the cells are not rinsed after contacting them with the cationic lipid. Preferably, the use of RPMI media is be avoided.

Oligomers were diluted separately to 10X the final desired concentration in Opti-MEM (without antibiotics) to a concentration of about 200 nM. Usually, a range from about 100 nM to about 4000 nM is used.

The partially diluted cationic lipid and the partially diluted oligomer were combined and mixed by inversion. The mixture was allowed to sit for about 10-15 minutes to allow complexing to occur. After complexing, pre-warmed growth medium was added to the cells. When using GSV, preferably the medium comprises serum and when using other lipids [(e.g., Perfect Lipids (available from In Vitrogen) or Lipofectin (available from Gibco BRL)] preferably, the medium comprises Opti-MEM reduced serum media. For in vitro transfection of cells, the use of media without antibiotics is preferred.

The cells can be in contact with the cationic lipid-oligomer composition for as long as 16 hours to thirty days with no toxic effects to the cells.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An oligomer comprising: an RNase H activating region and at least one nonactivating region, wherein the nonactivating region of the oligomer comprises at least one nucleomonomer having a 2′ OH propargyl group, said oligomer being sufficiently stabilized against nucleases.

2. The oligomer of claim 1, further comprising 5′ and 3′ termini which are stabilized against exonucleases.

3. The oligomer of claim 1, wherein the oligomer is about 15-40 nucleomonomers in length.

4. A chimeric antisense oligomer comprising a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 5′ terminal nucleomonomer is attached to an RNase H-activating region of between about three and ten contiguous phosphorothioate-linked nucleomonomers comprising deoxyribose, and wherein the 3′ terminus of said oligonucleotide is selected from the group consisting of: an inverted nucleomonomer, a contiguous stretch of about one to three phosphorothioate 2′-modified nucleomonomers, a biotin group, and a P-alkyloxyphosphotriester linked nucleomonomer, said oligomer having at least one nucleomonomer comprising a 2′ OH propargyl group.

5. A chimeric antisense oligomer comprising a 5′ terminus; a 3′ terminus; and 5′→3′ linked nucleomonomers independently selected from the group consisting of: 2′-modified phosphodiester linked nucleomonomers, and 2′-modified P-alkyloxyphosphotriester linked nucleomonomers; and wherein said 3′ terminal nucleomonomer is attached to an RNase H-activating region of between about three and ten contiguous phosphorothioate-linked nucleomonomers comprising deoxyribose, and wherein the 5′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester nucleomonomer, said oligomer having at least one nucleomonomer comprising a 2′ OH propargyl group.

6. A chimeric oligomer comprising: a 5′ terminus and a 3′ terminus, an RNase H activating region, and at least one nonactivating region, wherein a nonactivating region comprises at least one unmodified RNA ribonucleotide selected from the group consisting of: adenosine and guanine, said oligomer being sufficiently stabilized against nucleases.

7. A chimeric oligomer comprising: a 5′ terminus and a 3′ terminus, an RNase H activating region, and at least one nonactivating region, wherein a nonactivating region comprises a stretch between about 5 and about 10 of contiguous unmodified RNA ribonucleotides selected from the group consisting of: adenosine and guanine, said oligomer being sufficiently stabilized against nucleases.

8. A chimeric antisense oligomer comprising a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 5′ terminal nucleomonomer is attached to an RNase H-activating region of between about three and ten contiguous phosphorothioate-linked nucleomonomers comprising deoxyribose, and wherein the 3′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester linked nucleomonomer said oligomer comprising a stretch of contiguous unmodified RNA nucleomonomers selected from the group consisting of: adenosine and guanine, said oligomer being sufficiently stabilized against nucleases.

9. A chimeric antisense oligomers comprising: a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 3′ terminal nucleomonomer is attached to an RNase H-activating region of between about three and ten contiguous phosphorothioate-linked nucleomonomers comprising deoxyribose, and wherein the 5′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester linked nucleomonomer said oligomer comprising a stretch of contiguous unmodified RNA nucleomonomers selected from the group consisting of: adenosine and guanine, said oligomer being sufficiently stabilized against nucleases.

10. An oligomer comprising: an RNase H activating region, at least one nonactivating region, and at least one affinity enhancing agent, wherein said affinity enhancing agent is not positioned adjacent to an RNase H activating region, said oligomer being sufficiently stabilized against nucleases.

11. A chimeric antisense oligomer comprising a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 5′ terminal nucleomonomer is attached to an RNase H-activating region of between about three and ten contiguous phosphorothioate-linked nucleomonomers comprising deoxyribose, and wherein the 3′ terminus of said oligonucleotide is selected from the group consisting of: an inverted nucleomonomer, a contiguous stretch of one to three phosphorothioate linked 2′-modified nucleomonomers, a biotin group, and a P-alkyloxyphosphotriester linked nucleomonomer, said oligomer comprising at least one affinity enhancing agent, wherein said affinity enhancing agent is not positioned adjacent to an RNase H activating region.

12. A chimeric antisense oligomer comprising a 5′ terminus; a 3′ terminus; and 5′→3′ linked nucleomonomers independently selected from the group consisting of 2′-modified phosphodiester linked nucleomonomers, and 2′-modified P-alkyloxyphosphotriester linked nucleomonomers; and wherein said 3′ terminal nucleomonomer is attached to an RNase H-activating region of between about three and ten contiguous phosphorothioate-linked nucleomonomers comprising deoxyribose, and wherein the 5′ terminus of said oligonucleotide is selected from the group consisting of: an inverted nucleomonomer, a contiguous stretch of about one to three phosphorothioate linked 2′-modified nucleomonomers, a biotin group, and a P-alkyloxyphosphotriester linked nucleomonomer, said oligomer comprising at least one affinity enhancing agent, wherein said affinity enhancing agent is not positioned adjacent to an RNase H activating region.

13. A composition for inhibiting the expression of a protein in a cell comprising: an oligomer of any of cliams 1, 7, or 10 linked to a transporting peptide.

14. The composition of claim 13, wherein the transporting peptide comprises a peptide selected from the group consisting of: an active portion of the antennapedia protein, an active portion of the transportan protein, and an active portion of the HIV TAT protein.

15. A method for inhibiting the expression of a protein in a cell comprising contacting a cell with an oligomer of any of claims 1, 7, or 10 such that protein expression in the cell is inhibited.

16. A method for delivering an oligomer to a cell comprising contacting a cell with a mixture comprising an oligomer of any of claims 1, 7, or 10 and a cationic lipid for at least about three days such that an oligomer is delivered to a cell.
